(19) United States
(12) Patent Application Publication
(10) Pub. No.: US 2014/0350477 A1
(43) Pub. Date: Nov. 27, 2014

(54) DISPOSABLE SYRINGE

(71) Applicants: Young-Hee LEE, Gwangju (KR); LK MEDICAL CO. LTD., Hwasun-gun (KR)

(72) Inventor: Young-Hee Lee, Gwangju (KR)

(21) Appl. No.: 14/366,123

(22) PCT Filed: Jan. 18, 2013
(86) PCT No.: PCT/KR2013/000434

§ 371 Date: Jun. 17, 2014

(30) Foreign Application Priority Data
Jan. 20, 2012 (KR) 20-2012-0000550
Oct. 2, 2012 (KR) 10-2012-0109717

(51) Int. Cl. A61M 5/34

(52) U.S. Cl. CPC A61M 5/346
USPC 604/189; 604/218

(57) ABSTRACT

A disposable syringe including: a syringe cylinder (100) for containing a medicinal liquid therein, in which a medicinal liquid nozzle (120) is formed as a protrusion at a front end of the syringe cylinder, and a piston (110) is provided inside the syringe cylinder; and a medicinal liquid injection hub (200) including a needle (210) and a needle hub (220) and combined with the medicinal liquid nozzle in a removable manner, wherein a combining part (300) is provided in the medicinal liquid nozzle (120) and in the needle hub (220) so as to prevent a leakage of the medicinal liquid and to allow the needle hub to be both easily separated from and easily combined with the medicinal liquid nozzle by a user when using the syringe; without allowing the needle hub to be undesirably separated from the medicinal liquid nozzle.

### TECHNICAL FIELD

The present invention relates to a disposable syringe used to inject a patient with a medicinal liquid (medicinal injection, antibiotics and the like), in which a medicinal liquid injection hub, including a needle and a needle hub, can be easily separated from and easily combined with a medicinal liquid nozzle formed on the front end of a syringe cylinder; and, once the medicinal liquid injection hub is combined with the nozzle, a sealing effect is maintained, thereby preventing a leakage of the medicinal liquid from the syringe cylinder to the outside.

### BACKGROUND ART

Conventionally used disposable syringes are classified into a slim type syringe shown in FIG. 1 and a luer lock type syringe shown in FIG. 2. Due to safety problems experienced when using the slim type disposable syringes, the luer lock type disposable syringes may be preferred to the slim type disposable syringes.

However, the luer lock type disposable syringes are expensive and are inconvenient to users when using the syringes, so the slim type disposable syringes are more widely used than the luer lock type disposable syringes.

A conventional slim type disposable syringe which is more widely used as described above includes: a syringe cylinder for containing a medicinal liquid therein, a medicinal liquid nozzle protruding from an end of the syringe cylinder to a predetermined length and having a small diameter aperture and used to draw and discharge a medicinal liquid into and from the syringe cylinder, and a medicinal liquid injection hub combined with the medicinal liquid nozzle. For realizing prevention of infection, the slim type disposable syringe is typically made of a disposable plastic material (polypropylene, PP).

Described in detail, the slim type disposable syringe includes: a syringe cylinder 12 for containing a medicinal liquid therein, a piston 13 inserted into the syringe cylinder 12 through an open end of the cylinder and drawing a medicinal liquid into the syringe cylinder 12 or discharging the liquid from the cylinder by reciprocating in the cylinder. The outer circumferential surface of the syringe cylinder 12 is marked with degrees 16 for measuring the amount of medicinal liquid contained in the cylinder.

A medicinal liquid nozzle 14 having a small diameter aperture is formed in the slim type disposable syringe by protruding from the other end of the syringe cylinder 12 opposite to the open end to a predetermined length, so a medicinal liquid injection hub 15 can be combined with the medicinal liquid nozzle 14.

The medicinal liquid injection hub 15 combined with the medicinal liquid nozzle 14 of the syringe cylinder 12 includes: a needle 17 that is made of metal and has a sharp end designed to be inserted into the body a patient to a predetermined depth, and a needle hub 17a that holds the needle 17 and is combined with the medicinal liquid nozzle 14 of the syringe cylinder 12.

Further, to protect the needle 17 and to allow a user to safely handle the needle 17, the medicinal liquid injection hub 15 having the needle 17 in the front part thereof is covered with a protective cap 18.

When delivering the slim type disposable syringe 10 having the above-mentioned construction to a user, the syringe 10 may be packed in one package in which the medicinal liquid injection hub 15 covered with the protective cap 18 has been combined with the medicinal liquid nozzle 14 of the syringe cylinder 12; or may be packed in one package in which the syringe cylinder 12 and the medicinal liquid injection hub 15 are separated from each other; or may be packed in which the syringe cylinder 12 and the medicinal liquid injection hub 15 are separately packed in respective packages.

During a process of preparing a medicinal liquid prior to injecting a patient with the medicinal liquid using the slim type disposable syringe 10 delivered as described above, a doctor or a nurse (hereinbelow, referred to simply as “user”) may separate the medicinal liquid injection hub 15 from the syringe cylinder 10 or may combine the medicinal liquid injection hub 15 with the syringe cylinder 10.

Here, when the slim type disposable syringe is delivered in a packed state in which the medicinal liquid injection hub 15 covered with the protective cap has been combined with the medicinal liquid nozzle 14 of the syringe cylinder 12, the needle hub 17a is being only forcibly fitted over the medicinal liquid nozzle 14 without forming a construction capable of maintaining firm and sealed combination between the needle hub 17a and the medicinal liquid nozzle 14. Accordingly, when a user manipulates the syringe with intent to separate only the protective cap 18 from the medicinal liquid injection hub 15 or to separate both the protective cap 18 and the medicinal liquid injection hub 15 from the medicinal liquid nozzle 14, the manipulation results may become different from user's intentions. In other words, although the user intends to separate only the protective cap from the medicinal liquid injection hub; both the protective cap and the medicinal liquid injection hub may be separated from the medicinal liquid nozzle, or although the user intends to separate both the protective cap and the medicinal liquid injection hub from medicinal liquid nozzle, only the protective cap may be separated from the medicinal liquid injection hub. In the above case, the user should combine the elements with each other prior to separating the elements again as required.

The above-mentioned problems are frequently experienced when using the slim type disposable syringes, so, when intending to remove the protective cap 18 or the medicinal liquid injection hub 15, many users remove the protective cap while inconveniently holding the needle hub 17a with their fingers.

Further, when a user applies a force to the piston so as to inject a patient with a viscous medicinal liquid, the medicinal liquid injection hub 15 may be undesirably separated from the syringe cylinder 12 and the medicinal liquid may be undesirably discharged from the cylinder to the external environment. To prevent this problem, a user inconveniently injects a patient with a liquid while holding the medicinal liquid injection hub 15 fitted over the medicinal liquid nozzle 14 with a hand. Further, when a user produces a solution by dissolving powdered medicine of a vial in water for injection using the syringe, gas may be generated from the solution and may separate the medicinal liquid injection hub 15 from the syringe cylinder 12, so the solution may be discharged to and thus contaminating the surroundings; thus doctors and nurses, including the user, may come into contact with both or either of the gas and/or medicine.

Further, when it is necessary to inject a patient with a medicinal liquid using the syringe 10 combined with a 3-way stopcock, a user forcibly inserts the medicinal liquid nozzle of the syringe into a branch hose of the 3-way stopcock and injects the patient with the medicinal liquid while holding the syringe cylinder with a hand. However, in this case, when the user applies a force to the piston so as to inject the medicinal liquid, the medicinal liquid nozzle of the syringe may be undesirably separated from the branch hose of the 3-way stopcock. To prevent the medicinal liquid nozzle of the syringe from being undesirably separated from the branch hose of the 3-way stopcock during a process of injecting the medicinal liquid, it may be required for a doctor or a nurse to firmly hold the junction between the branch hose of the 3-way stopcock and the medicinal liquid nozzle of the syringe with a hand opposite to the hand holding the syringe cylinder.

An example of the luer lock type disposable syringes configured to solve the problems experienced in the slim type disposable syringes is shown in FIG. 2. The luer lock type disposable syringe is problematic in that the syringe is marketed in a packed state in which the syringe cylinder 12 and the medicinal liquid injection hub 15 are separately packed, so, prior to using the syringe, a user should combine the medicinal liquid injection hub 15 with the syringe cylinder 12. Another problem of the luer lock type disposable syringe is caused by the structure of the syringe in which an additional cylindrical wall is formed so as to surround the outer circumferential surface of the medicinal liquid nozzle 14, so the appearance of the medicinal liquid nozzle 14 becomes blunt and the size thereof is increased. Further, in this syringe, the medicinal liquid nozzle 14 should be located along the central axis of the syringe cylinder, so the use of the syringe having the enlarged medicinal liquid nozzle 14 is limited.

Further, when discarding the luer lock type disposable syringe after injecting using the syringe, it is required to separate the medicinal liquid injection hub 15 from the medicinal liquid nozzle 14 of the syringe cylinder 12. Here, the luer lock type disposable syringe is problematic in that, to separate the medicinal liquid injection hub 15 from the syringe cylinder 10, the protective cap 18 should be fitted over the medicinal liquid injection hub 15 and the medicinal liquid injection hub 15 covered with the protective cap should be rotated. However, during a process of covering the medicinal liquid injection hub 15 with the protective cap 18, a user may be pricked by the needle 17 and may become infected.

### DISCLOSURE

### Technical Problem

Accordingly, the present invention has been made keeping in mind the above problems occurring in the conventional syringes, and an object of the present invention is to provide a disposable syringe, which can efficiently supply a medicinal liquid (medicinal injection, antibiotics and the like) when injecting a patient with the medicinal liquid, and which can prevent a leakage of the medicinal liquid from the syringe to the outside when injecting the medicinal liquid, and in which a medicinal liquid injection hub can be easily and efficiently combined with and separated from a medicinal liquid nozzle of the syringe cylinder as a user intends, without being undesirably separated from the medicinal liquid nozzle, and which can prevent a user from being infected when discarding the used syringe.

### Technical Solution

In order to accomplish the above object, the present invention provides a disposable syringe, including: a syringe cylinder (100) for containing a medicinal liquid therein, in which a medicinal liquid nozzle is formed as a protrusion at a front end of the cylinder, and a piston is provided inside the cylinder; and a medicinal liquid injection hub including a needle and a needle hub and combined with the medicinal liquid nozzle in a removable manner, wherein

a combining part is provided in the medicinal liquid nozzle and in the needle hub so as to prevent a leakage of the medicinal liquid and to allow the needle hub to be easily separated from and easily combined with the medicinal liquid nozzle by a user when using the syringe, without allowing the needle hub to be undesirably separated from the medicinal liquid nozzle.

The combining part may include: a key formed on one of the medicinal liquid nozzle and the needle hub, and a keyhole formed in a remaining one of the medicinal liquid nozzle and the needle hub so as to be engaged with the key, wherein a key seat is formed by continuously extending from an end of the keyhole at a predetermined angle relative to the keyhole. Alternatively, the combining part may include: protruding threads formed in one of the medicinal liquid nozzle and the needle hub, and depressed threads formed in a remaining one of the medicinal liquid nozzle and the needle hub so as to be engaged with the protruding threads.

The medicinal liquid nozzle may be configured such that the outer diameter thereof becomes gradually reduced within a range from a central portion to an outside end thereof, and the needle hub may be configured such that the inner diameter thereof becomes gradually reduced within a range from a central portion to an end thereof.

Further, the medicinal liquid injection hub may be configured such that the needle is integrated with a front end of the needle hub, and the medicinal liquid injection hub may be a branch hose of a 3-way stopcock C, and the syringe cylinder may be marked with degrees for measuring the amount of medicinal liquid.

### Advantageous Effects

As described above, the disposable syringe according to the present invention can efficiently supply a medicinal liquid (medicinal injection, antibiotics and the like) when injecting a patient with the medicinal liquid, and can prevent a leakage of the medicinal liquid from the syringe to the outside when injecting the medicinal liquid, and allows a medicinal liquid injection hub to be easily and efficiently combined with and separated from a medicinal liquid nozzle of the syringe cylinder as a user intends, without allowing the medicinal liquid injection hub to be undesirably separated from the medicinal liquid nozzle, and can prevent a user from being infected when discarding the used syringe.

### DESCRIPTION OF DRAWINGS

FIG. 1 is an exploded perspective view of a syringe according to the first embodiment of the related art;

FIG. 2 is an exploded perspective view of a syringe according to the second embodiment of the related art;

FIG. 3 is a perspective view of an important part of a disposable syringe according to the first embodiment of the present invention;

FIG. 4 is a perspective view of an important part of a disposable syringe according to the second embodiment of the present invention;

FIG. 5 is a perspective view of an important part of a disposable syringe according to the third embodiment of the present invention;

FIG. 6 is a perspective view of an important part of a disposable syringe according to the fourth embodiment of the present invention;

FIG. 7 is a perspective view illustrating an embodiment of the use of a disposable syringe according to the present invention; and

FIG. 8 is a perspective view illustrating another embodiment of the use of a disposable syringe according to the present invention.

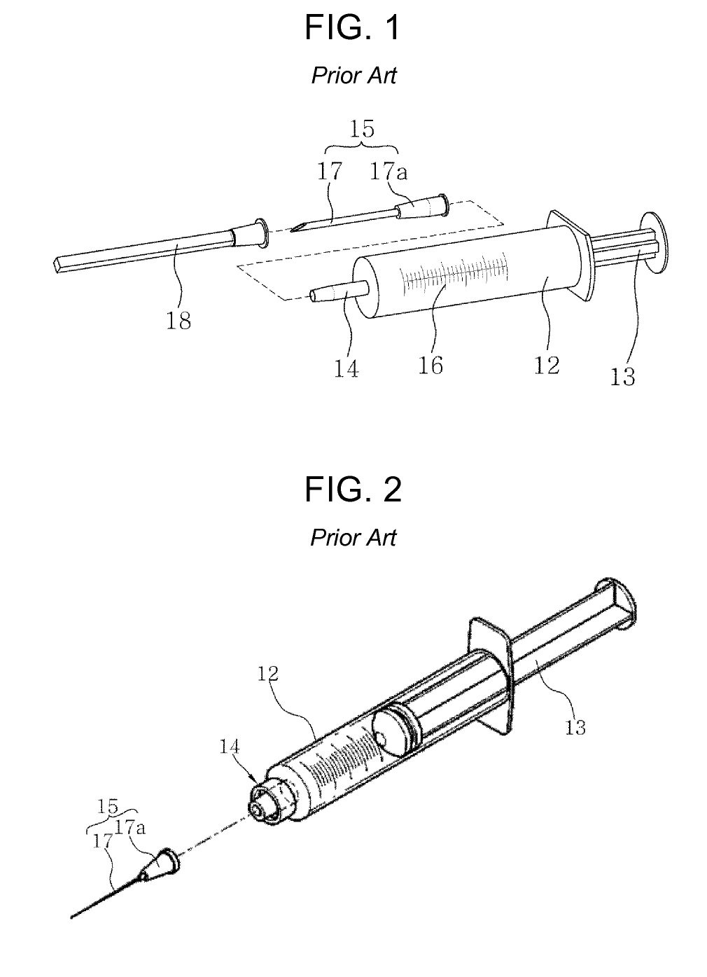

### BEST MODE

Hereinbelow, a disposable syringe according to the present invention will be described in detail with reference to the accompanying drawings.

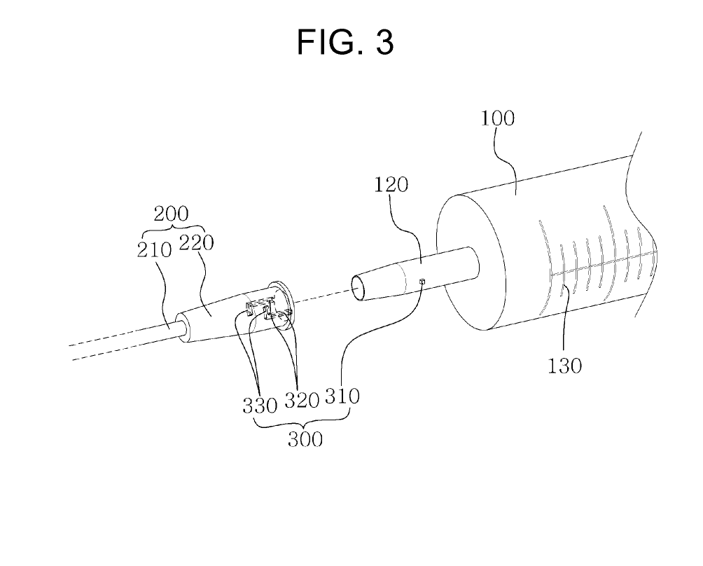

As shown in FIGS. 3 to 6, the disposable syringe A according to the present invention includes: a syringe cylinder 100 for containing a medicinal liquid therein, in which a medicinal liquid nozzle 120 is formed as a protrusion at the front end of the cylinder, and a piston 110 is provided inside the cylinder; and a medicinal liquid injection hub 200 including a needle 210 and a needle hub 220 and combined with the medicinal liquid nozzle in a removable manner, wherein

a combining part 300 is provided in the medicinal liquid nozzle 120 and in the needle hub 220, so as to prevent a leakage of the medicinal liquid and to allow the needle hub to be easily separated from and easily combined with the medicinal liquid nozzle by a user when using the syringe, without allowing the needle hub to be undesirably separated from the medicinal liquid nozzle.

As shown in FIG. 3, the combining part 300 according to the first embodiment comprises a key 310 formed on the medicinal liquid nozzle 120, and a keyhole 320 formed inside the needle hub 220 of the medicinal liquid injection hub 200 so as to be engaged with the key 310, wherein a key seat 330 is formed by continuously extending from an end of the keyhole 320 at a predetermined angle relative to the keyhole 320.

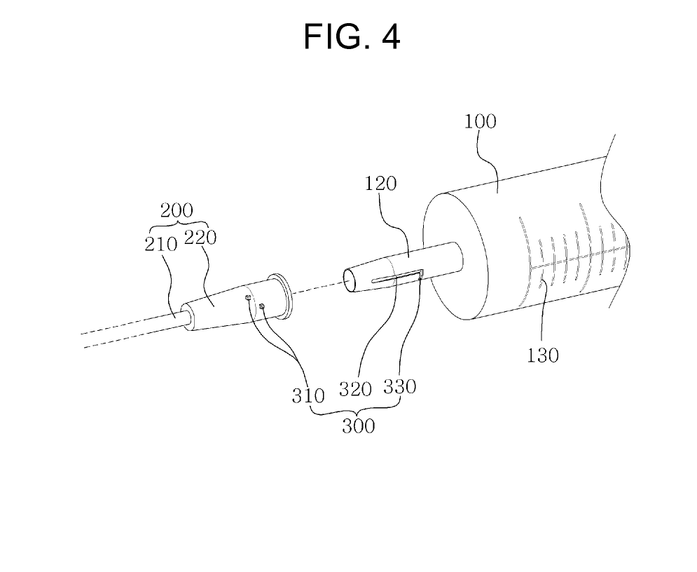

As shown in FIG. 4, the combining part 300 according to the second embodiment of the present invention comprises a key 310 formed inside the needle hub 220 of the medicinal liquid injection hub 200, and a keyhole 320 formed on the medicinal liquid nozzle 120 so as to be engaged with the key 310, wherein a key seat 330 is formed by continuously extending from an end of the keyhole 320 at a predetermined angle relative to the keyhole 320.

To combine the needle hub 220 with the medicinal liquid nozzle 120 using the combining part 300 according to one of the first and second embodiments of the present invention, the key 310 is aligned with the inlet of the keyhole 320, and then the key 310 is inserted into the keyhole 320. Thereafter, the medicinal liquid nozzle 120 or the medicinal liquid injection hub 200 which is provided with the key 310 is rotated in a direction toward the key seat 330, so the key 310 is seated into the key seat 330, thereby being firmly and stably engaged with the key seat 330.

Here, the medicinal liquid nozzle 120 is configured such that the outer diameter thereof becomes gradually reduced within a range from a central portion to an outside end thereof, and the needle hub 220 of the medicinal liquid injection hub 200 is configured such that the inner diameter thereof becomes gradually reduced within a range from a central portion to an end thereof.

The gradual reduction in the inner and outer diameters of the elements is to realize a sealed combination between the medicinal liquid nozzle 120 and the needle hub 200 and to realize a desired sealing effect in the junction between them. Here, the medicinal liquid nozzle 120 and the needle hub 220 are made of a plastic material or a resin material which has a predetermined elasticity, so the medicinal liquid nozzle 120 and the needle hub 220 can be firmly combined with each other such that desired water tightness can be formed in the junction between them.

Once the key 310 is seated into the key seat 330 after passing through the keyhole 320, the key 310 is prevented from being undesirably removed from the key seat 330, so the medicinal liquid nozzle 120 and the needle hub 220 can be brought into firm and stable combination.

Further, the combination between the medicinal liquid nozzle 120 and the needle hub 220 is realized using the key 310, so it is easy and simple to combine or separate the needle hub 220 with and from the medicinal liquid nozzle 120, thereby solving the problems experienced in the conventional syringes.

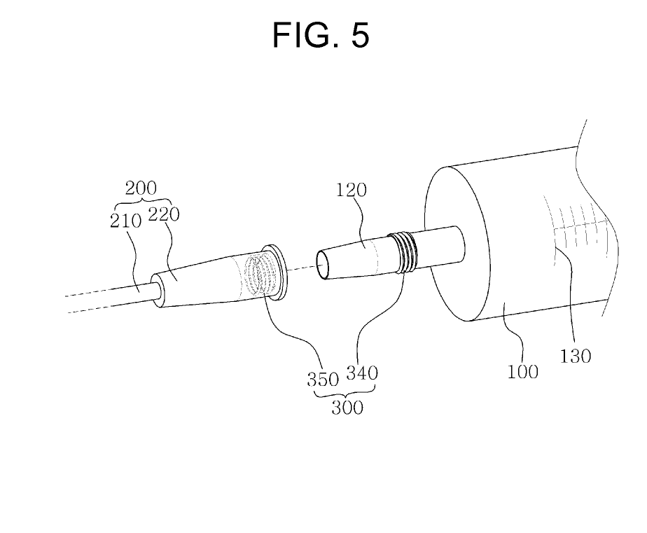

As shown in FIG. 5, the combining part 300 according to the third embodiment comprises protruding threads 340 formed around the outer circumferential surface of the medicinal liquid nozzle 120, and depressed threads 350 formed around the inner circumferential surface of the needle hub 220 of the medicinal liquid injection hub 200 so as to be engaged with the protruding threads 340, such that the combination between the medicinal liquid nozzle 120 and the needle hub 220 can be realized by an engagement between the protruding threads 340 and the depressed threads 350.

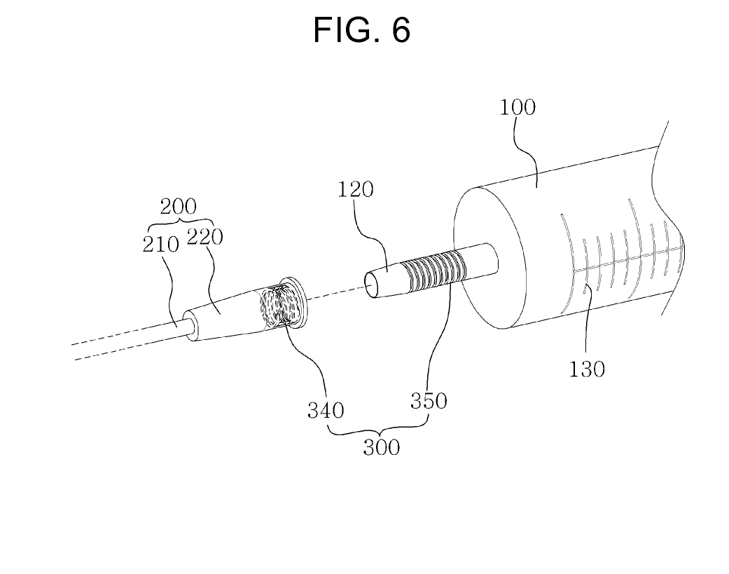

As shown in FIG. 6, the combining part 300 according to the fourth embodiment comprises protruding threads 340 formed around the inner circumferential surface of the needle hub 220 of the medicinal liquid injection hub 200, and depressed threads 350 formed around the outer circumferential surface of the medicinal liquid nozzle 120 so as to be engaged with the protruding threads 340, so the combination between the medicinal liquid nozzle 120 and the needle hub 220 can be realized by an engagement between the protruding threads 340 and the depressed threads 350.

To combine the needle hub 220 with the medicinal liquid nozzle 120 using the combining part 300 according to one of the third and fourth embodiments of the present invention, the protruding threads 340 are engaged with the depressed threads 350, thereby realizing firm and stable combination between the needle hub 220 and the medicinal liquid nozzle 120. Further, once the needle hub 220 is combined with the medicinal liquid nozzle 120 by the engagement of the threads, the needle hub 220 can be prevented from being undesirably separated from the medicinal liquid nozzle 120 and desired water tightness can be formed in the junction between them.

Here, the medicinal liquid nozzle 120 is configured such that the outer diameter thereof becomes gradually reduced within a range from a central portion to an outside end thereof, and the needle hub 220 is configured such that the inner diameter thereof becomes gradually reduced within a range from a central portion to an end thereof.

The gradual reduction in the inner and outer diameters of the elements is to realize a sealed combination between the medicinal liquid nozzle 120 and the needle hub 200 and to realize a desired sealing effect in the junction between them. Here, the medicinal liquid nozzle 120 and the needle hub 220 are made of a plastic material or a resin material which has predetermined elasticity, so the medicinal liquid nozzle 120 and the needle hub 220 can be firmly combined with each other such that desired water tightness can be formed in the junction between them.

Further, the combination between the medicinal liquid nozzle 120 and the needle hub 220 is realized by an engagement between the protruding threads 340 and the depressed threads 350, so it is easy and simple to combine or separate the needle hub 220 with and from the medicinal liquid nozzle 120, thereby solving the problems experienced in the conventional syringes.

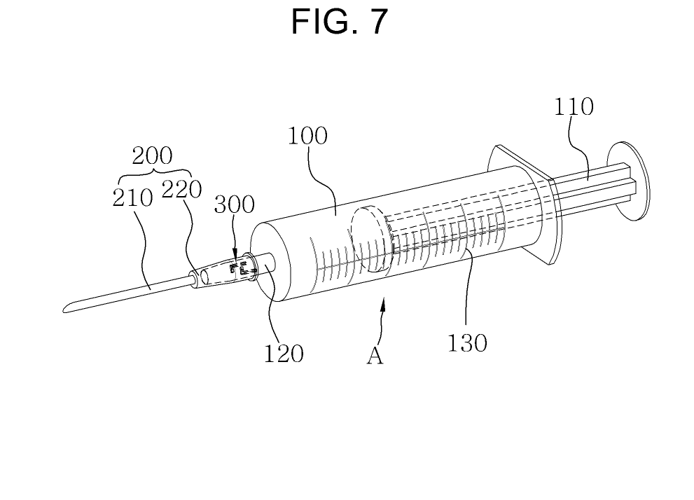

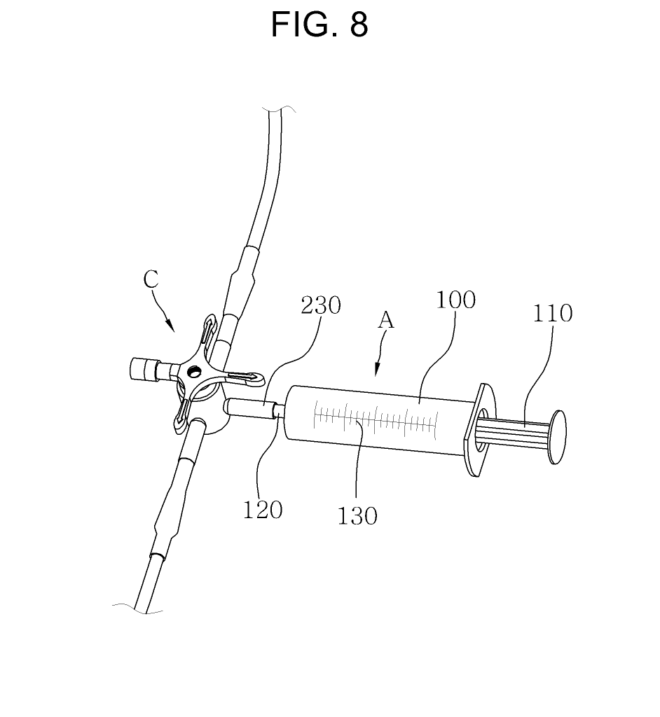

Further, the medicinal liquid injection hub 200 according to the present invention may comprise a needle hub 220 and a needle 210 that is configured to be directly pierced into the body of a patient as shown in FIG. 7, or may take the form of a branch hose 230 that is configured to supply a medicinal liquid from the syringe cylinder 100 to another unit, such as a 3-way stopcock C, as shown in FIG. 8.

To realize desired water tightness in the junction between the branch hose 230 and the medicinal liquid nozzle 120 after combining them, the branch hose 230 may be configured such that the inner diameter thereof becomes gradually reduced within a range from a central portion to an end thereof, and the medicinal liquid nozzle 120 may be configured such that the outer diameter thereof becomes gradually reduced within a range from a central portion to an outside end thereof.

Further, to combine the branch hose 230 with the medicinal liquid nozzle 120, the combining part according to one of the first to fourth embodiments shown in FIGS. 3 to 6 may be used, so the branch hose 230 can be easily and simply combined with and separated from the medicinal liquid nozzle 120.

As described above, the disposable syringe according to the present invention can efficiently supply a medicinal liquid (medicinal injection, antibiotics and the like) when injecting a patient with the medicinal liquid, and can prevent a leakage of the medicinal liquid from the syringe to the outside when injecting the medicinal liquid, and allows a medicinal liquid injection hub to be easily and efficiently combined with and separated from a medicinal liquid nozzle of the syringe cylinder as a user intends, without allowing the medicinal liquid injection hub to be undesirably separated from the medicinal liquid nozzle.

1. A disposable syringe (A), comprising: a syringe cylinder (100) for containing a medicinal liquid therein, in which a medicinal liquid nozzle (120) is formed as a protrusion at a front end of the cylinder, and a piston (110) is provided inside the cylinder; and a medicinal liquid injection hub (200) including a needle (210) and a needle hub (220) and combined with the medicinal liquid nozzle in a removable manner, wherein
a combining part (300) is provided in the medicinal liquid nozzle (120) and in the needle hub (220) so as to prevent a leakage of the medicinal liquid and to allow the needle hub to be easily separated from and easily combined with the medicinal liquid nozzle by a user when using the syringe, without allowing the needle hub to be undesirably separated from the medicinal liquid nozzle.

2. The disposable syringe of claim 1, wherein
the combining part (300) comprises: a key (310) formed on one of the medicinal liquid nozzle and the needle hub, and a keyhole (320) formed in a remaining one of the medicinal liquid nozzle and the needle hub so as to be engaged with the key, in which a key seat (330) is formed by continuously extending from an end of the keyhole at a predetermined angle relative to the keyhole.

3. The disposable syringe of claim 1, wherein
the combining part (300) comprises: protruding threads (340) formed in one of the medicinal liquid nozzle and the needle hub, and depressed threads (350) formed in a remaining one of the medicinal liquid nozzle and the needle hub so as to be engaged with the protruding threads.

4. The disposable syringe of claim 1, wherein
the medicinal liquid nozzle (120) is configured such that an outer diameter thereof becomes gradually reduced within a range from a central portion to an outside end thereof, and the needle hub is configured such that an inner diameter thereof becomes gradually reduced within a range from a central portion to an end thereof.

5. The disposable syringe of claim 1, wherein
the medicinal liquid injection hub (200) is configured such that the needle is integrated with a front end of the needle hub.

6. The disposable syringe of claim 1, wherein
the medicinal liquid injection hub (200) is a branch hose (230) of a 3-way stopcock (C).

7. The disposable syringe of claim 1, wherein
the syringe cylinder (100) is marked with degrees (130) for measuring an amount of medicinal liquid.
